(19) United States
(12) Patent Application Publication
(10) Pub. No.: US 2008/0063698 A1
(43) Pub. Date: Mar. 13, 2008

(54) Crystallization inhibition of drugs in transdermal drug delivery systems and methods of use

(75) Inventor: Rod Lawson Hartwig, Plantation, FL (US)

Correspondence Address: Noven Pharmaceuticals, Inc.;Jay G. Kolman, Esq., 11960 S.W. 144 Street, Miami, FL, 33186, US

(73) Assignee: NOVEN PHARMACEUTICALS, INC.

(21) Appl. No.: 11/897,189

(22) Filed: Aug. 29, 2007

Related U.S. Application Data
(63) Continuation of application No. 10/751,152, filed on Jan. 2, 2004, now abandoned.
Application No. 10/751,152 is a continuation of application No. 10/353,624, filed on Jan. 29, 2003, now abandoned.
Application No. 10/353,624 is a continuation of application No. 10/010,342, filed on Dec. 5, 2001, now abandoned.
(60) Provisional application No. 60/251,294, filed on Dec. 5, 2000.

(51) Int. Cl. A61K 31/56; A61F 13/00; A61P 43/00; A61F 13/02

(52) U.S. Cl. 424/448; 424/449; 514/170

(57) ABSTRACT

The invention relates to compositions and methods for making a transdermal drug delivery system capable of achieving substantially zero-order kinetics for delivery of the active agent over a period of time in excess of 24 hours and at least 72 hours, comprising a pharmaceutically acceptable active agent carrier and a rosin ester which provides a crystal inhibiting and drug stabilizing effect on the active agents incorporated therein.

### CROSS-REFERENCE TO RELATED APPLICATION

This application is based on and claims the benefit of Provisional Application 60/251,294 filed Dec. 5, 2000, which is incorporated in its entirety herein by reference.

### BACKGROUND OF THE INVENTION

This invention relates generally to transdermal drug delivery systems, and more particularly to pressure-sensitive adhesive compositions, that incorporate rosin esters to inhibit crystal formation of the active agent in the composition during storage.

The use of transdermal drug delivery systems as a means to topically administer an active agent is well known. Such systems incorporate the active agent into a carrier composition, such as a polymeric and/or pressure-sensitive adhesive composition, from which the active agent is delivered through the skin or mucosa of the user.

In general, transdermal drug delivery systems are either reservoir-type or matrix-type. Both types of systems include a backing layer that forms the protective outer surface of the finished transdermal device and which is exposed to the environment during use, and a release liner or protective layer that forms the inner surface and which covers the adhesive means for affixing the device to the skin or mucosa of a user. The release liner or protective layer is removed prior to application, exposing the adhesive means, which is typically a pressure-sensitive adhesive. The active agent is located between the release liner and backing layer, usually solubilized or dispersed in a solvent or carrier composition.

In a reservoir-type device, the active agent, typically in fluid or gel form, is isolated from the adhesive means used to affix the device to the user. Traditionally, a reservoir system referred to a device having a pocket or “reservoir” which served to hold the active agent and which was formed in or by the backing layer itself. A peripheral adhesive layer was then used to affix the device to the user. While such devices are still in use today, the term reservoir has become known as a device which employs one or more permeable layers, such as rate controlling membranes and drug permeable adhesives layers, laminated over the reservoir (which is typically nothing more than another layer containing the drug in a carrier composition), in order to more effectively control the delivery rate of the active agent and attachment of the device to the user.

A matrix-type device generally comprises the active agent solubilized or dispersed in an adhesive carrier composition, typically a pressure-sensitive adhesive or bioadhesive, which functions as both the drug carrier and the adhesive means of applying the system to the skin or mucosa. Such devices are described, for example, in U.S. Pat. Nos. 4,994,267, 5,446,070, 5,474,783 and 5,656,286, all of which are assigned to Noven Pharmaceuticals, Inc., Miami, Fla.

A particular advantage over other forms of drug delivery, such as oral administration, is that the transdermal system can provide a continuous and controlled release of the active agent over a prolonged period of time so that the resulting blood levels remain constant.

It has been shown that the degree of saturation and solubility of the active agent in the carrier composition are determining factors in controlling delivery of the active agent from the transdermal system. Since only solubilized active agent is available for delivery out of the transdermal system, the carrier composition must not promote crystal growth or formation, especially during storage of the system prior to use. Generally, active agents have been found to be readily soluble in acrylic polymers. However, in order to deliver a therapeutically effective amount to the system's user, and to also achieve the desired adhesive strength required for topical application in a matrix-type system, additional polymers and ingredients are often added to the carrier composition (for example, incorporating a rubber, polysiloxane or polyvinylpyrrolidone polymer). Such additional polymers and ingredients can affect the recrystallization of the active agent in the carrier composition. The tendency for crystal formation or growth is known, for example, in the case of high melting point hydrophobic drugs, such as hormones and steroidal active agents, which tend to be poorly soluble or insoluble in pressure-sensitive adhesive carrier compositions because they form strong crystal bonds.

Formulation of transdermal systems is further frequently hampered by poor solubility of certain active agents in the carrier composition, which in turn also severely limits its therapeutic application. This formulating aspect is particularly difficult in matrix-type systems because the carrier composition has to be optimized not only to incorporate and administer the desired active agents, but also to obtain sufficient wear properties (means of attachment to the user) for the adhesive carrier. While using low concentrations in order to incorporate the active agent into the carrier may not deleteriously affect the carrier's adhesive properties, low active agent concentration can result in difficulties in achieving an acceptable delivery rate. Poor or inadequate solubility of the active agent further gives rise to crystal formation or growth.

Generally, concentrations of the active agent substantially at or near the saturation solubility, and even supersaturated (i.e., an amount of active agent at a concentration greater than the solubility of the active agent in the carrier composition at room temperature) are sought in order to increase or maximize delivery rates. Such systems are also desirable because they provide the ability to potentially achieve continuous administration of the active drug in therapeutically effective amounts for prolonged periods of time, such as greater than 24 hours, and even up to 7 days or more. In these systems, however, the active agent can more easily recrystallize, especially during storage. Crystallization may occur after a few weeks or months of storage. This gives rise to stability problems.

Active agent that is present in crystalline form cannot be delivered through skin or mucosa. Inadequate delivery of the active agent in turn leads to blood levels falling below that which are therapeutically effective. Some transdermal systems rely upon both solubilized and crystalline forms of active agent to achieve the desired drug loading in the carrier composition. Although the drug crystals in such systems are intended to dissolve later, for example after application, such a process is unpredictable and interferes with achieving a controlled delivery rate, especially a zero-order kinetic delivery rate.

Failure to control crystal formation and growth can further interfere with the physical properties of the transdermal system. The presence of crystals, particularly in large amounts, can interfere with the carrier composition's adhesive properties in matrix-type transdermal systems. Furthermore, surface crystals can come into direct contact with the skin or mucosa and promote irritation. The presence of drug crystals is therefore generally undesirable.

To prevent crystallization in transdermal systems, compounds which in individual cases have been described in the art as crystallization inhibitors and/or used to improve the storage stability of transdermal systems include polyvinylpyrrolidone, cellulosic polymers, polyethylene oxide, polyvinyl alcohol, polyacrylic acid, gelatins, cyclodextrins, silica, silicon dioxide, starch (derivatives) and dextran.

It has been found that rosin esters, in particular wood rosin esters, are suitable to suppress or prevent crystal formation of active agents in transdermal systems, and additionally provide very good in vitro flux rates and delivery profiles, particularly with hydrophobic drugs. While use of rosin esters as tackifying agents in transdermal systems (i.e., to improve or impart tack properties to adhesive compositions) is known in the art, their use as crystallization inhibitors alone has not been described. U.S. Pat. No. 5,478,567 is particularly characterized by finding that when compounded with 1-menthol in a specified ratio, a rosin ester derivative will serve as a solubilizer for nonsteroidal antiphlogistic analgesic drugs. U.S. Pat. Nos. 5,885,612 and 6,143,319 are particularly characterized by the fact that the estrogen-containing pressure-sensitive adhesive itself is mainly composed of certain rosin esters, in amounts ranging from 50% to 92%, the first in conjunction with a styrene-isoprene block copolymer, the second in conjunction with ethyl cellulose.

### SUMMARY OF THE INVENTION

It is therefore an object of this invention to provide a transdermal drug delivery system that can substantially suppress or prevent crystallization of active agents incorporated therein.

It is another object of this invention to provide a transdermal drug delivery system that can substantially suppress or prevent crystallization formation or growth of the active agents incorporated in a pressure-sensitive adhesive carrier composition and delivery a therapeutically effective amount while retaining good physical adhesive properties.

It is also an object of this invention to provide a transdermal drug delivery system that can incorporate the drug substantially at saturated and supersaturated concentrations of the active agent, and deliver the same at a controlled and predictable release rate.

It is a further object of this invention to provide for transdermal drug delivery systems that can incorporate active agents that are insoluble or sparingly soluble in pressure-sensitive adhesives in amounts necessary to deliver a therapeutically effective amount without resulting in recrystallization of the active agent after a few weeks or months of storage, and deliver the same at a controlled and predictable release rate.

It is still another object of this invention to provide a method for increasing the solubilizing and stabilizing of active agents in transdermal delivery systems.

It is additionally an object of this invention to provide a method for making a transdermal drug delivery system that achieves a substantially zero-order kinetic rate of drug delivery for a prolonged period of time without crystallization of the active agent therein.

### BRIEF DESCRIPTION OF DRAWINGS

FIG. 1 is a schematic illustration of a matrix-type transdermal drug delivery system of the present invention.

FIG. 2 is a graphical representation of the cumulative flux rate of methyltestosterone through cadaver skin from pressure-sensitive adhesive carrier compositions of the present invention comprising rosin esters as compared to a pressure-sensitive adhesive carrier composition comprising polyvinylpyrrolidone.

FIG. 3 is a graphical representation of the cumulative flux rate of methyltestosterone through cadaver skin from pressure-sensitive adhesive carrier compositions of the present invention comprising varying concentrations of rosin esters.

FIG. 4 illustrates the type of delivery kinetics which can be achieved from an adhesive carrier composition of the present invention comprising methyltestosterone. The graph demonstrates the extended duration of the substantially zero-order delivery from the transdermal system.

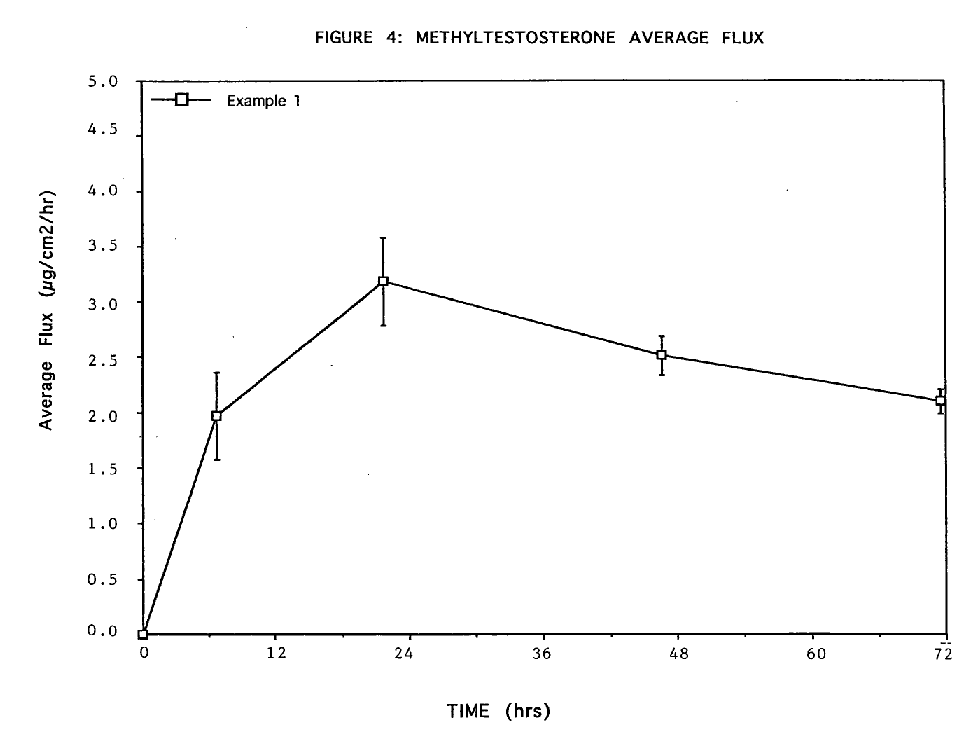

### DETAILED DESCRIPTION OF THE INVENTION

The foregoing and other objects are achieved by the present invention which provides transdermal drug delivery systems containing rosin esters as crystallization inhibitors for the active agents incorporated into the carrier composition.

The term “topical” or “topically” is used herein in its conventional meaning as referring to direct contact with an anatomical site or surface area on a mammal including skin, teeth, nails and mucosa.

The term “mucosa” as used herein means any moist anatomical membrane or surface on a mammal such as oral, buccal, vaginal, rectal, nasal or ophthalmic surfaces.

The term “transdermal” as used herein means passage of an active agent into and/or through skin or mucosa for localized or systemic delivery.

The term “solubilized” is intended to mean that in the carrier composition there is an intimate dispersion or dissolution of the active agent at the crystalline, molecular or ionic level, such that crystals of the active agent cannot be detected using a microscope having a magnification of 25×. As such, the active agent is considered herein to be in “non-crystallized” form when in the compositions of the present invention.

As used herein, the term “flux” is defined as the absorption of the drug through the skin or mucosa, and is described by Fick's first law of diffusion:
J=−D(dCm/dx),
Where J is the flux in g/cm2/sec, D is the diffusion coefficient of the drug through the skin or mucosa in cm2/sec and Dcm/dx is the concentration gradient of the drug across the skin or mucosa.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

To prevent crystal formation in transdermal systems during storage and to be able to administer therapeutically effective amounts of active agents, rosin esters are added to transdermal drug delivery systems according to the invention. By the addition of rosin esters, the active agent is able to remain solubilized during storage. Moreover, transdermal systems containing rosin esters are able to accommodate more active agent while still demonstrating very good in vitro flux rates.

The rosin esters suitable for transdermal systems according to the present invention include pentaerythritol esters of hydrogenated wood rosin, e.g. FORAL® 105; glycerol esters of hydrogenated wood rosin, e.g. FORAL® 85; pentaerythritol esters of partially hydrogenated wood rosin, e.g. FORALYN® 110, PENTALYN® H-E; pentaerythritol esters of wood rosin, e.g. PENTALYN® A, PERMALYN® 5110, 6110, 5135; pentaerythritol esters of modified wood rosin, e.g. PENTALYN® G, X and 856; glycerol esters of partially hydrogenated wood rosin, e.g. STAYBELITE® Ester 5 and 10; triethylene glycol esters of hydrogenated rosin, e.g. STAYBELITE® Ester 3; glycerol esters of partially dimerized rosin, e.g., POLY-PALE® Ester 10 and HERCULES® Ester Gum 10D; pentaerythritol esters of tall oil rosin, e.g. PERMALYN® 505 and 3100, HERCULES® Ester Gum 8D-SP; glycerol esters of tall oil rosin, e.g. PERMALYN® 2085; pentaerythritol esters of dimerized rosin, e.g. PENTALYN® K; pentaerythritol esters of partially dimerized rosin, e.g. PENTALYN® C; and similar rosins from Hercules, Inc., and combinations and mixtures thereof.

In accordance with one aspect of the invention, an improved pressure-sensitive adhesive carrier composition which is suitable for delivery of an active agent from a matrix-type transdermal system comprises one or more pressure-sensitive adhesives and a rosin ester.

Preferred rosin esters are pentaerythritol esters. Pentaerythritol rosin esters can be prepared by any known technique in the art or can be obtained commercially (for example, from Hercules, Inc., Wilmington, Del.). The particularly preferred pentaerythritol esters are of wood rosins such as those commercially available under the name PENTALYN® and PERMALYN®.

Pentaerythritol esters of wood rosin are particularly suitable to suppress or prevent crystallization in pressure-sensitive adhesive carrier compositions containing hormones and steroidal active agents such as methyltestosterone, and allow for the delivery of a desired dose continuously.

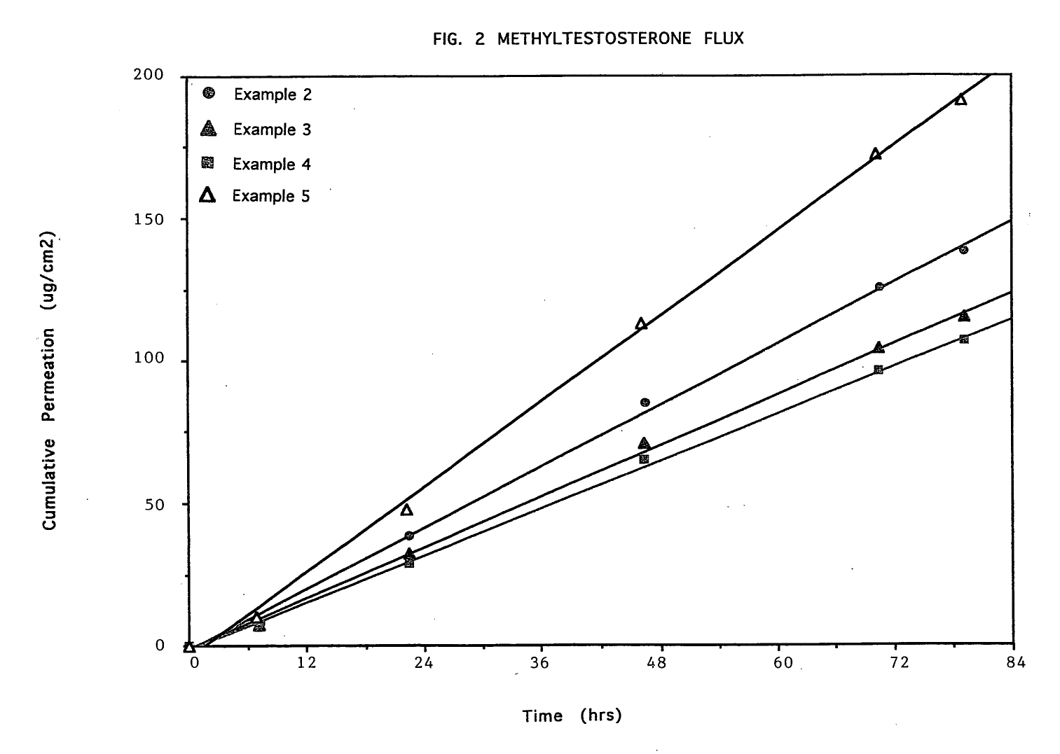

FIG. 2 graphically demonstrates the in vitro flux results through cadaver skin from matrix-type transdermal systems comprising pressure-sensitive carrier compositions with and without rosin esters or soluble polyvinylpyrrolidone (PVP). The use of soluble PVP as a drug crystallization inhibitor and solubility enhancer is known in the art.

The four formulations were prepared using the method of Example I to yield compositions having the ingredient concentrations, by weight based on the dry weight of the total carrier composition, as set forth in TABLE I.

The flux results set forth in FIG. 2 were conducted within three weeks after preparation and prior to the observation of any crystals as seen in TABLE II.

As seen in FIG. 2, the adhesive carrier compositions containing either a rosin ester or polyvinylpyrrolidone, or both, flux at relatively the same rates. The adhesive carrier composition without a rosin ester, however, formed crystals which would ultimately cause the flux rate to be decreased upon topical application to a user. And while the adhesive carrier composition containing both a rosin ester and a polyvinylpyrrolidone fluxes at the lowest rate, which is expected since the drug solubility of the composition increases (i.e., the drug delivery forces are decreased), the carrier composition is then able to incorporate more drug and yield a flux rate (Example 5) which is greater.

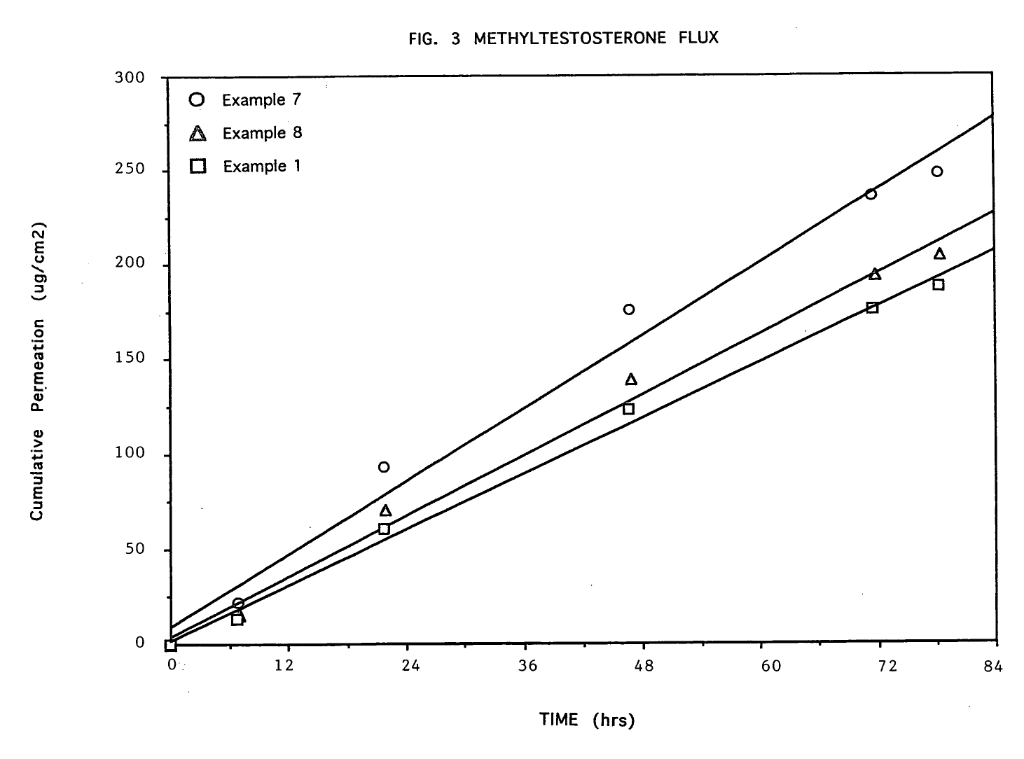

This effect is further demonstrated as seen in FIG. 3 when the concentration of the rosin ester increases. As the rosin ester concentration increases, crystal inhibition of the active agent increases (as shown in TABLE III) while the flux somewhat decreases.

In accordance with another aspect of the invention, an improved carrier composition which is suitable for delivery of an active agent from a transdermal system comprises a rosin ester and one or more other crystallization inhibitors, and in particular polyvinylpyrollidone.

The term “polyvinylpyrrolidone” or “PVP” refers to a polymer, either a homopolymer or copolymer, containing vinylpyrrolidone (also referred to as N-vinylpyrrolidone, N-vinyl-2-pyrrolidone and N-vinyl-2-pyrrolidinone) as a monomeric unit. PVP polymers include soluble and insoluble homopolymeric PVPs, and copolymers such as vinylpyrrolidone/vinyl acetate and vinylpyrrolidone/dimethylamino-ethylmethacrylate. The cross-linked homopolymer is insoluble and is generally known in the pharmaceutical industry under the designations polyvinylpolypyrrolidone, crospovidone and PVP. The copolymer vinylpyrrolidone-vinyl acetate is generally known in the pharmaceutical industry under the designations Copolyvidon(e), Copolyvidonum or VP-VAc.

The term “soluble when used with reference to PVP means that the polymer is soluble in water and generally is not substantially cross-linked, and has a molecular weight of less than about 2,000,000. See, generally, Buhler, KOLLIDON®: POLYVINYLPYRROLIDONE FOR THE PHARMACEUTICAL INDUSTRY, BASF Aktiengesellschaft (1992). Soluble PVP polymers have been identified in the pharmaceutical industry under a variety of names, the most commonly used include Povidone, Polyvidon(e), Polyvidonum, Polyvidonum, poly(N-vinyl-2-pyrrolidinone, poly(N-vinylbutyrolactam), poly(1-vinyl-2-pyrrolidone), poly[1-(2-oxo-1-pyrrolidinyl)ethylene].

The amount and type of PVP required in the preferred embodiments will depend on the quantity and type of drug present in the adhesive composition, as well as the type of adhesives, but can be readily determined through routine experimentation.

Typically, the PVP is present in an amount from about 1% to about 25% by weight, preferably from about 1% to about 20% by weight of the dry weight of the total adhesive carrier composition.

Said PVP preferably has a molecular weight of about 2,000 to 1,200,000, more preferably 5,000 to 100,000, and most preferably 7,000 to 54,000. PVP having a molecular weight of about 1,000,000 to about 1,500,000 is also preferred.

PVPs are sold to the pharmaceutical industry under the trademarks KOLLIDON by BASF AG, Ludwigshafen, Germany; PLASDONE, POLYPLASDONE and COPOLYMER 958 by ISP Technologies, Wayne, N.J. Preferred PVPs are KOLLIDON 12PF, 17PF, 25, 30, 90 and VA-64.

The amount and type of rosin ester required in the practice of the invention will depend on the one or more additional polymeric materials and ingredients in the carrier composition, and on the amount and type of active agent. Generally, the amount of rosin ester to be used is an amount sufficient to deliver a therapeutically effective amount of the active agent at a substantially zero-order kinetic rate of delivery for a prolonged period of time (i.e., greater than 24 hours), and to substantially suppress or prevent recrystallization of the active agent during storage. Typically, the amount of rosin ester to be used ranges from about 0.5% to about 25%, preferably from about 1.0% to 20%, and more preferably from about 1.0% to 15% by weight based on the dry weight of the total carrier composition.

As used herein, “therapeutically effective” means an amount of an active agent that is sufficient to achieve the desired local or systemic effect or result, such as to prevent, cure, diagnose, mitigate or treat a disease or condition, when applied topically over the duration of intended use. The amounts necessary are known in the literature or may be determined by methods known in the art, but typically range from about 0.1 mg to about 20,000 mg, and preferably from about 0.1 mg to about 1,000 mg, and most preferably from about 0.1 to about 500 mg per human adult or mammal of about 75 kg body weight per 24 hours.

The term “active agent” (and its equivalents “agent,” “drug,” “medicament” and “pharmaceutical”) is intended to have the broadest meaning and includes at least one of any therapeutic, prophylactic, pharmacological or physiological active substance, cosmetic and personal care preparations, and mixtures thereof, which is delivered to a mammal to produce a desired, usually beneficial, effect. More specifically, any active agent that is capable of producing a pharmacological response, localized or systemic, irrespective of whether therapeutic, diagnostic, cosmetic or prophylactic in nature, is within the contemplation of the invention. It should be noted that the active agents can be used singularly or in combinations and mixtures.

There is no limitation on the type of active agent that can be used in this invention. However, active agents that are solid at room temperature are preferred.

The active agents contained in the carrier composition can be in different forms depending on the solubility and release characteristics desired, for example as neutral molecules, components of molecular complexes, and pharmaceutically acceptable salts, free acids or bases, or quaternary salts of the same. Simple derivatives of the drugs such as pharmaceutically acceptable ethers, esters, amides and the like which have desirable retention and release characteristics but which are easily metabolized at body pH, and enzymes, pro-active forms, pro-drugs and the like, can also be employed.

Hormones and steroidal active agents, natural and synthetic, that generally tend to be poorly soluble or insoluble in pressure-sensitive adhesive carrier compositions are preferred and include, for example, Estrogenically effective steroid hormones such as Colpormon, Conjugated Estrogens, Estradiol (17β- and α-) and its Esters (e.g., Acetate, Benzoate, Cypionate, Dipropionate Diacetate, Enanthate, Undecylate and Valerate), Estriol, Estrone, Ethinyl Estradiol, Equilenin, Equilin, Mestranol, Moxestrol, Mytatrienediol, Quinestradiol and Quinestrol; Progestagenically effective steroid hormones such as Allylestrenol, Anagestone, Chlormadinone Acetate, Delmadinone Acetate, Demegestone, Desogestrel, 3-Keto Desogestrel, Dimethisterone, Dydrogesterone, Ethinylestrenol, Ethisterone, Ethynodiol (and Diacetate), Flurogestone Acetate, Gestodene, Gestonorone Caproate, Haloprogesterone, (17-Hydroxy- and 17-Acetate-) 16-Methylene-Progesterone, 17α-Hydroxyprogesterone (Acetate and Caproate), Levonorgestrel, Lynestrenol, Medrogestone, Medroxyprogesterone (and Acetate), Megestrol Acetate, Melengestrol, Norethindrone (Acetate and Enanthate), Norethisterone, Norethynodrel, Norgesterone, Norgestimate, Norgestrel, Norgestrienone, 19-Norprogesterone, Norvinisterone, Pentagestrone, Progesterone, Promegestone, Quingestrone and Trengestone; Androgenically effective steroid hormones such as Aldosterone, Androsterone, Boldenone, Cloxotestosterone, Dehydroepiandrosterone, Fluoxymesterone, Mestanolone, Mesterolone, Methandrostenolone, Methyltestosterone, 17α-Methyltesteosterone, 17α-Methyltestosterone 3-Cyclopentyl Enol Ether, Norethandrolone, Normethandrone, Oxandrolone, Oxymesterone, Oxymetholone, Prasterone, Stanlolone, Stanozolol, Testosterone (Acetate, Enanthate, Isobutyrate, Propionate and Undecanoate), Testosterone 17-Chloral Hemiacetal, Testosterone 17β-Cypionate and Tiomesterone.

Other specific drugs for which rosin esters can be particularly usefully employed according to the invention include:

1. α-Adrenergic Agonist agents such as Phenylpropanolamine and Talipexole.

2. Analgesics and/or Anti-Migraine such as Acetaminophen, Acetylsalicylic Acid, Buprenorphine, Codeine, Fentanyl, Hydomorphone, Lisuride, Salicylic Acid derivatives, Sufentanil and Sumatriptan.

3. Anti-Allergic agents such as Amlexanox, Astemizole, Azelastine, Cromolyn, Fenpiprane, Ibudilast, Nedocromil, Oxatomide, Pentigetide, Repirinast, Tranilast and Traxanox.

4. Anesthetic agents such as Benzocaine, Bupivicaine, Cocaine, Dibucaine, Dyclonine, Etidocaine, Lidocaine, Mepivacaine, Prilocaine, Procaine and Tetracaine.

5. Anoretic agents such as Fenfluramine, Mazindol and Phentermine.

6. Anti-Bacterial (antibiotic) agents including Aminoglycosides, β-Lactams, Cephamycins, Macrolides, Penicillins, Polypeptides and Tetracyclines.

7. Anti-Cancer agents such as Aminolevulinic Acid, 5-Fluouracil, Methotrexate, Tamoxifen and Taxol.

8. Anti-Cholinergic agents such as Atropine, Eucatropine and Procyclidine.

9. Anti-Diabetic agents such as Glipizide, Glyburide, Glypinamide, Insulins, Repaglinide, Rosiglitazone and Troglitazone.

10. Anti-Emetic agents such as Acetylleucine Monoethanolamine, Alizapride, Benzquinamide, Bietanautine, Bromopride, Buclizine, Chlorpromazine, Clebopride, Cyclizine, Dimenhydrinate, Dipheniodol, Domperidone, Granisetron, Meclizine, Methalltal, Metoclopramide, Metopimazine, Nabilone, Ondansteron, Oxypendyl, Pipamazine, Piprinhydrinate, Prochlorperazine, Scopolamine, Tetrahydrocannabinols, Thiethylperazine, Thioproperzaine, Trimethobenzamide and Tropisetron.

11. Anti-Fungal agents such as Clortrimazole, Ketoconazole, Miconazole, Nystatin and Triacetin.

12. Antihistamine agents such as Tricyclics such as Ahistan, Etymemazine, Fenethazine, N-Hydroxyethylpromethazine Chloride, Isopromethazine, Mequitazine, Promethazine, Pyrathiazine, and Thiazinamium Methyl Sulfate, and Loratadine and Clobenzepam.

13. Anti-Hyperlipoproteinemic agents such as Atorvastatin, Cerivastatin, Lovastatin, Pravastatin and Simvastatin.

14. Anti-Hyperthyroid agents such as Methimazole.

15. Anti-Inflammatory and/or Corticoid agents such as Beclomethasone, Betamethasone (and Acetate, Diproprionate and Valerate), Corticosterone, Cortisone, Deoxycortocosterone (and Acetate), Dexamethasone, Diclofenac, Fenoprofen, Flucinolone (and Acetonide), Fludrocortisone, Fluocinonide, Flunisolide, Fluradrenolide, Flurbiprofen, Halcinonide, Hydrocortisone (and Acetate), Ibuprofen, Ibuproxam, Indoprofen, Ketoprofen, Ketorolac, Naproxen, Oxametacine, Oxyphenbutazone, Piroxicam, Prednisolone, Prednisone, Suprofen and Triamcinolone (and Acetonide).

16. Anti-Malarial agents such as Pyrimethamine.

17. Anti-Parkinson's and/or Anti-Alzhiemer's agents such as Biperiden, Bromocriptine, Cabergoline, 1-Hydroxy-Tacrine, Levodopa, Lisuride, Pergolide, Pramipexole, Quinpirole, Ropinirole, Rivastigmine, Physostigimine, Selegiline (Deprenyl and L-Deprenyl), Tacrine and Teruride.

18. Anti-Psychotic and/or Anti-Anxiety and/or Anti-Depressant agents such as Acetophenazine, Bromperidol, Chlorproethazine, Chlorpromazine, Clomipramine, Clozapine, Fluoxetine, Fluphenazine, Haloperidol, Loxapine, Mesoridazine, Molindone, Paroxetine, Perphenazine, Piperacetazine, Sertraline, Thiopropazate, Thioridazine, Thiothixene, Trifluoperazine, Triflupromazine and Venlafaxine.

19. Anti-Ulcerative agents such as Enprostil and Misoprostol.

20. Anti-Viral agents such as Acyclovir, Rimantadine and Vidarabine.

21. Anxiolytic agents such as Azapirones such as Buspirone and Ipsapirone, Benzodiazepines such as Alprazolam, Chlordiazepoxide, Clonazepam, Clorazepate, Diazepam, Flurazepam, Halazepam, Lorazepam, Oxazepam, Oxazolam, Prazepam and Triazolam.

22. β-Adrenergic agonist agents such as Albuterol, Carbuterol, Fenoterol, Metaproterenol, Mirtazapine, Rimiterol, Quinterenol, Salmefamol, Soterenol, Tratoquinol, Terbutaline and Terbuterol.

23. Bronchodilators such as Azelastine, and Ephedrine derivatives including Epiniphrine and Isoproterenol, Albuterol, Salbutanol, Clenbuterol and Theophylline.

24. Cardioactive agents such as Atenolol, Benzydroflumethiazide, Bendroflumethiazide, Calcitonin, Captopril, Chlorothiazide, Clonidine, Clopamide, Dobutamine, Dopamine, Diltiazem, Enalapril, Enalaprilat, Gallopamil, Indomethacin, Isosorbide (Dinitrate and Mononitrate), Monoxidil, Nicardipine, Nifedipine, Nitroglycerin, Papaverine, Prazosin, Procainamide, Propranolol, Prostaglandin (E₁ and E₂), Quinidine Sulfate, Timolol, and Verapamil.

25. Central Nervous System stimulants and agents such as Dextroamphetamine, Methylphenidate (and each Enantiomer and Free Base Form) and Nicotine.

26. Cholinergic agents such as Acetylcholine, Arecoline, Bethanechol, Carbachol, Choline, Methacoline, Muscarine and Pilocarpine.

27. Muscle relaxants such as Baclofen and Cyclobenzaprine.

28. Narcotic antagonist agents such Nalmfene and Naloxone.

The amount of active agent to be incorporated in the carrier composition will vary depending on the particular active agent, the desired therapeutic effect, and the time span for which the transdermal system is to provide therapy. Normally, the amount of active agent in the transdermal system can vary from about 0.1% to about 50%, and preferably from about 0.1% to about 30% by weight based on the dry weight of the total carrier composition. For lower dose concentrations permitted by this invention, such as with steroidal hormones, the preferred amount is from about 0.1% to about 10%, and more preferably from about 0.1% to about 6%.

While not essential, it is further preferred that the drug, and in particular steroids and hormones, most particularly androgenic hormones, be incorporated substantially at or near and even above saturation with respect to their concentration in the carrier composition rather than substantially at subsaturation.

The term “carrier” as used herein refers to any non-aqueous material known in the art as suitable for transdermal drug delivery administration, and includes any polymeric material into which an active agent may be solubilized in combination or admixture with the other ingredients of the composition. The polymeric materials preferably comprise adhesives and, in particular, pressure-sensitive adhesives. The carrier material is typically used in an amount of about 10% to about 90%, and preferably from about 10% to about 75%, by weight based on the dry weight of the total carrier composition.

The term “carrier composition” may also refer to enhancers, solvents, co-solvents and other types of additives useful for facilitating transdermal drug delivery. An “adhesive” as used herein means any natural or synthetic substance that is capable of surface attachment to the topical site of the transdermal drug delivery system.

Wood rosin esters have been found to be highly effective in preventing crystallization of active agents in pressure-sensitive adhesive carrier compositions. An adhesive is a pressure-sensitive adhesive within the meaning of the term as used herein if it has the properties of a pressure-sensitive adhesive per se or if it functions as a pressure-sensitive adhesive by admixture with tackifiers, plasticizers, cross-linking agents or other additives.

Pressure-sensitive adhesives include all of the non-toxic natural and synthetic polymers known or suitable for use in transdermal systems as adhesives, such as polyacrylates, polysiloxanes, silicones, rubbers, gums, polyisobutylenes, polyvinylethers, polyurethanes, styrene block copolymers, styrene/butadiene polymers, polyether block amide copolymers, ethylene/vinyl acetate copolymers, and vinyl acetate based adhesives.

The pressure-sensitive adhesives particularly useful in practicing, this invention include polyacrylates of one or more monomers of acrylic acids or other copolymerizable monomers. Polyacrylate adhesives also include polymers of alkyl acrylates and/or methacrylates and/or copolymerizable secondary monomers, or monomers with functional groups, and in particular non-hydroxy functional groups. The term “polyacrylate” is intended to be used interchangeably with the terms acrylic, acrylate and polyacrylic as used herein and as known in the art. Suitable pressure-sensitive acrylic adhesives are commercially available and include those sold under the trademark DURO-TAK® by National Starch and Chemical Company, Bridgewater, N.J., and GELVA® Multipolymer Solution by Solutia, Inc., St. Louis, Mo.

The pressure-sensitive adhesives useful in practicing the invention include solvent-based, hot melt and grafted adhesives, and may be used alone or in combinations, mixtures or blends. Particularly preferred blends include blends of polyacrylates and polysiloxanes.

The carrier compositions of the present invention can also contain one or more solvents and/or co-solvents. Such solvents and/or co-solvents are those known in the art, and are non-toxic, pharmaceutically acceptable substances, preferably liquids, which do not substantially negatively affect the adhesive properties or the solubility of the active agents at the concentrations used. The solvent and/or co-solvent can be for the active agent or for the carrier materials, or both.

Suitable solvents include volatile liquids such as alcohols (e.g., methyl, ethyl, isopropyl alcohols and methylene chloride); ketones (e.g., acetone); aromatic hydrocarbons such as benzene derivatives (e.g., xylenes and toluenes); lower molecular weight alkanes and cycloalkanes (e.g., hexanes, heptanes and cyclohexanes); and alkanoic acid esters (e.g., ethyl acetate, n-propyl acetate, isobutyl acetate, n-butyl acetate isobutyl isobutyrate, hexyl acetate, 2-ethylhexyl acetate or butyl acetate); and combinations and mixtures thereof.

Suitable co-solvents include polyhydric alcohols, which include glycols, triols and polyols such as ethylene glycol, diethylene glycol, propylene glycol, dipropylene glycol, trimethylene glycol, butylene glycol, polyethylene glycol, hexylene glycol, polyoxethylene, glycerin, trimethylpropane, sorbitol, polyvinylpyrrolidone, and the like.

Further suitable co-solvents include glycol ethers such as ethylene glycol monoethyl ether, glycol esters, glycol ether esters such as ethylene glycol monoethyl ether acetate and ethylene glycol diacetate; saturated and unsaturated fatty acids, mineral oil, silicone fluid, lecithin, retinol derivatives and the like, and ethers, esters and alcohols of fatty acids.

Although the exact amount of co-solvents that may be used in the carrier composition depends on the nature and amount of the other ingredients, such amount typically ranges from about 0.1% to about 40%, and preferably from about 0.1% to about 30% by weight, and more preferably from about 1% to about 20%, by weight based on the dry weight of the total carrier composition.

In certain embodiments of the invention, an enhancer is incorporated into the carrier composition. The term “enhancers” as used herein refers to substances used to increase permeability and/or accelerate the delivery of an active agent through the skin or mucosa, and include monhydric alcohols such as ethyl, isopropyl, butyl and benzyl alcohols; or dihydric alcohols such as ethylene glycol, diethylene glycol, or propylene glycol dipropylene glycol and trimethylene glycol; or polyhydric alcohols such as glycerin, sorbitol and polyethylene glycol, which enhance drug solubility; polyethylene glycol ethers of aliphatic alcohols (such as cetyl, lauryl, oleyl and stearly) including polyoxyethylene (4) lauryl ether, polyoxyethylene (2) oleyl ether and polyoxyethylene (10) oleyl ether commercially available under the trademark BRIJ® 30, 93 and 97 from ICI Americas, Inc., and BRI® 35, 52, 56, 58, 72, 76, 78, 92, 96, 700 and 721; vegetable, animal and fish fats and oils such as cotton seed, corn, safflower, olive and castor oils, squalene, and lanolin; fatty acid esters such as propyl oleate, decyl oleate, isopropyl palmitate, glycol palmitate, glycol laurate, dodecyl myristate, isopropyl myristate and glycol stearate which enhance drug diffusibility; fatty acid alcohols such as oleyl alcohol and its derivatives; fatty acid amides such as oleamide and its derivatives; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered and esters of sorbitol and sorbitol anhydride such as polysorbate 20 commercially available under the trademark Tween® 20 from ICI Americas, Inc., as well as other polysorbates such as 21, 40, 60, 61, 65, 80, 81, and 85. Other suitable enhancers include oleic and linoleic acids, triacetin, ascorbic acid, panthenol, butylated hydroxytoluene, tocopherol, tocopherol acetate, tocopheryl linoleate. If enhancers are incorporated into the carrier composition, the amount typically ranges up to about 30%, and preferably from about 0.1% to about 15%, by weight based on the dry weight of the total carrier composition.

In addition to enhancers, there may also be incorporated various pharmaceutically acceptable additives and excipients available to those skilled in the art. These additives include tackifying agents such as aliphatic hydrocarbons, mixed aliphatic and aromatic hydrocarbons, aromatic hydrocarbons, substituted aromatic hydrocarbons, hydrogenated esters, polyterpenes, silicone fluid, mineral oil and hydrogenated wood rosins. Additional additives include binders such as lecithin which “bind” the other ingredients, or Theological agents (thickeners) containing silicone such as fumed silica, reagent grade sand, precipitated silica, amorphous silica, colloidal silicon dioxide, fused silica, silica gel, quartz and particulate siliceous materials commercially available as Syloid®, Cabosil®, Aerosile®, and Whitelite®, for purposes of enhancing the uniform consistency or continuous phase of the final composition. Other additives and excipients include diluents, stabilizers, fillers, clays, buffering agents, biocides, humectants, anti-irritants, antioxidants, preservatives, plasticizing agents, cross-linking agents, flavoring agents, colorants, pigments and the like. Such substances can be present in any amount sufficient to impart the desired properties to the carrier composition. Such additives or excipients are typically used in amounts up to 25%, and preferably from about 0.1% to about 10%, by weight based on the dry weight of the total carrier composition.

The carrier compositions according to the present invention can be prepared by first mixing appropriate amounts of the rosin esters in volatile polar and/or non-polar organic liquids such as those previously described as suitable volatile solvents. Appropriate amounts of active agent(s) are then added to the mixture together with appropriate amounts of pressure-sensitive adhesive(s), solvent(s) and/or co-solvent(s), with or without enhancer(s), and thoroughly blended. The mixture of the carrier composition is next formed into a film at ambient temperature, preferably by coating or casting at a controlled specified thickness onto a flexible sheet material, such as a release liner, followed by evaporation of the volatile solvents at elevated temperatures (e.g., by passing through an oven). The non-volatile or higher boiling point solvents and/or co-solvents, such as the polyols, used in the carrier composition remain therein. The carrier composition that has been coated or cast on the flexible sheet material, is then laminated to another flexible sheet material preferably a backing layer. Appropriate size and shape individual transdermal drug delivery systems are cut and then packaged (e.g., pouched).

The order of steps, the amount of the ingredients, and the amount and time of mixing may be important process variables which will depend on the specific polymers, active agents, solvents and/or co-solvents, enhancers and additives and excipients used in the composition. These factors can be adjusted by those skilled in the art, while keeping in mind the objects of achieving a solubilized active agent and providing a uniform product that will also give desirable results.

Further details and examples of pressure-sensitive adhesives, enhancers, solvents, co-solvents, and other additives, as well as transdermal systems generally, suitable in practicing the invention are described in U.S. Pat. Nos. 5,474,787, 5,656,286 and 60/115,987, all of which are assigned to Noven Pharmaceuticals, Inc. and incorporated herein by reference.

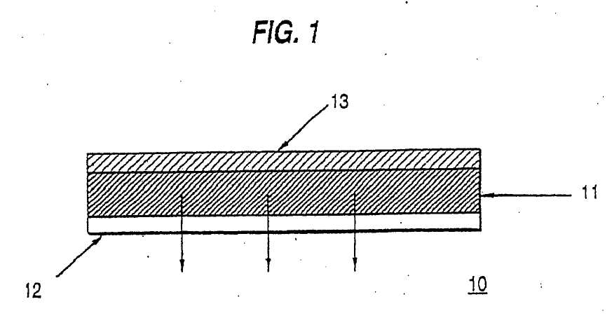

A particularly preferred structure for the transdermal drug delivery system useful in practicing this invention is a matrix-type system. Reference to FIG. 1 shows a matrix-type transdermal drug delivery system 10 comprising a pressure-sensitive adhesive carrier composition layer 11, a release liner 12, and a backing layer 13. Removal of the release liner 12 exposes the pressure-sensitive adhesive carrier composition for topical application to the user.

It is understood that a reservoir-type system, provided with a separate pressure-sensitive adhesive layer or adhesive means of attachment, is contemplated in practicing the invention and may well be of advantage in certain cases. The reservoir-type system may further consist of one or more layers or membranes. Regardless of the type of transdermal system used to practice the invention, the carrier composition is preferably non-aqueous (i.e., substantially free of water).

The phrase “substantially zero-order” as used herein means delivery of the active agent through the skin or mucosa at a rate which is approximately constant once steady state is attained. Typical variability contemplated within the scope of this meaning is about 30% to about 40% difference from the mean in blood levels of active agent at steady state (within about 24 hours after topical application).

### EXAMPLES

The above description and following specific examples are hereby illustrative of pharmaceutically acceptable active agent carrier compositions and transdermal drug delivery systems, and methods of making same, within the contemplation of the invention. The description and examples are in no way intended to be, or should be considered, limiting of the scope of the invention. And while efforts have been made to ensure accuracy with respect to numbers used (such as amounts and temperatures), some experimental error and deviation should be accounted for and/or allowed.

### Example 1

A methyltestosterone pressure-sensitive adhesive mixture was prepared by combining 37.3 parts of a polysiloxane adhesive (BIO-PSA® Q7-4603, a silicone pressure-sensitive adhesive in toluene; Dow Corning Corporation, Medical Products, Midland, Mich.), 2.3 parts of methyltestosterone, 6.1 parts polyvinylpyrrolidone (KOLLIDON® 30), 8.6 parts pentaerythritol ester of wood rosin (PENTALYN® A), 5.6 parts of toluene, 2.9 parts of isopropyl alcohol, 3.5 parts of oleic acid, 3.5 parts of dipropylene glycol, and 30.2 parts of a polyacrylate adhesive (GELVA® 3087, an acrylic pressure sensitive adhesive in ethyl acetate; Solutia, Inc., St. Louis, Mo.) were added and thoroughly mixed in an appropriate sized container until the polymer blend was uniform. The resulting composition had the ingredient concentrations on a dry weight basis (i.e. after solvent evaporation of volatile solvents) as shown below.

| INGREDIENT | WEIGHT % |
|---|---|
| Polysiloxane Adhesive | 39.0 |
| (BIO-PSA ® Q7-4603) | |
| Polyacrylate Adhesive | 20.0 |
| (GELVA ® 3087) | |
| Polyvinylpyrrolidone | 10.0 |
| (KOLLIDON ® 30) | |
| Wood Rosin | 15.0 |
| (PENTALYN ® A) | |
| Oleic Acid | 6.0 |
| Dipropylene Glycol | 6.0 |
| Methyltestosterone | 4.0 |
| | 100.0 |

### Examples 2-8

In the following examples, the method of Example 1 was used with the appropriate amounts of starting materials to yield compositions having the following ingredient concentrations set forth in tabular form in TABLE I.

TABLE I

| | WEIGHT % | | | | | | |
|---|---|---|---|---|---|---|---|
| INGREDIENT | Ex. 2 | Ex. 3 | Ex. 4 | Ex. 5 | Ex. 6 | Ex. 7 | Ex. 8 |
| Polysiloxane | 40.0 | 40.0 | 40.0 | 40.0 | 54.0 | 49.0 | 44.0 |
| Adhesive | | | | | | | |
| (BIO-PSA ® | | | | | | | |
| Q7-4603) | | | | | | | |
| Polyacrylate | 33.5 | 33.5 | 23.5 | 21.0 | 20.0 | 20.0 | 20.0 |
| Adhesive | | | | | | | |
| (GELVA ® 3087) | | | | | | | |
| Polyvinylpyrrolidone | 10.0 | — | 10.0 | 10.0 | 10.0 | 10.0 | 10.0 |
| (KOLLIDON ® 30) | | | | | | | |
| Wood Rosin Ester | — | 10.0 | 10.0 | 10.0 | — | 5.0 | 10.0 |
| (PENTALYN ® A) | | | | | | | |
| Oleic Acid | 6.0 | 6.0 | 6.0 | 6.0 | 6.0 | 6.0 | 6.0 |
| Dipropylene Glycol | 8.0 | 8.0 | 8.0 | 8.0 | 6.0 | 6.0 | 6.0 |
| Methyltestosterone | 2.5 | 2.5 | 2.5 | 5.0 | 4.0 | 4.0 | 4.0 |

The rate of crystal formation of the active agent in the matrix-type systems of Examples 2, 3 and 4, and 1, 6, 7 and 8 were compared and the appearance of drug crystal formation set forth in tabular form in TABLES II and III, respectively. The observations of crystal formation were done by visual inspection using a microscope having a magnification of 25×.

TABLE II
Crystal Formation

| Example | 1 Month | 2 Months |
|---|---|---|
| 2 | Very small branches at | Small branches throughout |
| | edges | |
| 3 | None | None |
| 4 | None | None |

TABLE III
Crystal Formation

| Example | 1 Month | 2 Months |
|---|---|---|
| 1 | None | None |
| 6 | Small chunks throughout | Loaded with large chunks |
| 7 | None | Many small chunks |
| 8 | None | Small chunks throughout |

Comparison of examples 2, 3 and 4 over a two month period set forth in TABLE II, maintained in aluminum foil at 25° C.±5° C., demonstrate the instability of the active agent in the adhesive carrier composition without the use of a wood resin when contrasted with the same carrier composition incorporating polyvinylpyrrolidone, a well known hormonal drug crystallization inhibitor and solubility enhancer.

Comparison of examples 1, 6, 7 and 8 over a two month period set forth in TABLE III demonstrates the distinct reduction in active agent crystal formation with increasing concentrations of rosin esters in the same adhesive carrier composition.

What is claimed is:

1. A composition for transdermal delivery of an active agent in a flexible, finite system comprising a blend of:
a therapeutically effective amount of one or more active agents;
a pharmaceutically acceptable carrier; and
a rosin ester in an amount up to 25% by weight of the total composition, said amount being sufficient to suppress crystal formation of the one or more active agents during storage of the system,
wherein the composition excludes 1-menthol and is capable of delivering a therapeutically effective amount of the one or more active agents at a substantially zero-order kinetic rate of delivery to the skin or mucosa of a patient in need thereof over a period in excess of 24 hours.

2. The composition according to claim 1, wherein the rosin ester is selected from the group consisting of pentaerythritol esters of hydrogenated wood rosin, glycerol esters of hydrogenated wood rosin, pentaerythritol esters of partially hydrogenated wood rosin, pentaerythritol esters of wood rosin, pentaerythritol esters of modified wood rosin, glycerol esters of partially hydrogenated wood rosin, triethylene glycol esters of hydrogenated rosin, glycerol esters of partially dimerized rosin, pentaerythritol esters of tall oil rosin, glycerol esters of tall oil rosin, pentaerythritol esters of dimerized rosin, pentaerythritol esters of partially dimerized rosin, and combinations and mixtures thereof.

3. The composition according to claim 2, wherein the rosin ester is a pentaerythritol ester.

4. The composition according to claim 3, wherein the pentaerythritol ester is an ester of a non-hydrogenated wood rosin.

5. The composition according to claim 2, wherein the pharmaceutically acceptable carrier is a pressure-sensitive adhesive.

6. The composition according to claim 5, wherein the pressure-sensitive adhesive carrier includes a non-hydroxy functional polyacrylate polymer.

7. The composition according to claim 6, further comprising a polyvinylpyrrolidone and an enhancer, and the active agent is an androgen selected from the group consisting of testosterone and methyltestosterone.

8. The composition according to claim 1, wherein the one or more active agents is selected from the group consisting of hormonal and steroidal active agents.

9. A composition for transdermal delivery of an active agent in a flexible, finite system comprising a blend of:
a therapeutically effective amount of one or more active agents;
a pharmaceutically acceptable pressure-sensitive adhesive carrier comprising a polyacrylate in an amount of about 15% to about 35% by weight of the total composition and a polysiloxane in an amount of about 35% to about 55% by weight of the total composition; and
a rosin ester in an amount up to 25% by weight of the total composition, said amount being sufficient to suppress crystal formation of the one or more active agents during storage of the system,
wherein the composition excludes 1-menthol and is capable of delivering a therapeutically effective amount of the one or more active agents at a substantially zero-order kinetic rate of delivery to the skin or mucosa of a patient in need thereof over a period of at least 72 hours.

10. The composition according to claim 9, wherein the rosin ester is selected from the group consisting of pentaerythritol esters of hydrogenated wood rosin, glycerol esters of hydrogenated wood rosin, pentaerythritol esters of partially hydrogenated wood rosin, pentaerythritol esters of wood rosin, pentaerythritol esters of modified wood rosin, glycerol esters of partially hydrogenated wood rosin, triethylene glycol esters of hydrogenated rosin, glycerol esters of partially dimerized rosin, pentaerythritol esters of tall oil rosin, glycerol esters of tall oil rosin, pentaerythritol esters of dimerized rosin, pentaerythritol esters of partially dimerized rosin, and combinations and mixtures thereof.

11. The composition according to claim 10, wherein the rosin ester is a pentaerythritol ester.

12. The composition according to claim 11, wherein the pentaerythritol ester is an ester of a non-hydrogenated wood rosin.

13. The composition according to claim 12, wherein the pressure-sensitive adhesive carrier includes a non-hydroxy functional polyacrylate polymer.

14. The composition according to claim 13, further comprising a polyvinylpyrrolidone and an enhancer.

15. The composition according to claim 14, wherein the one or more active agents is selected from the group consisting of hormonal and steroidal active agents.

16. The composition according to claim 15, wherein the steroidal active agent is an androgen selected from the group consisting of boldenone, fluoxymesterone, mestanolone, mesterolone, methandrostenolone, methyltestosterone, norethandrolone, normethandrone, oxandrolone, oxymesterone, oxymetholone, prasterone, stanlolone, stanozolol, testosterone, and tiomesterone.

17. The composition according to claim 16, wherein the androgen is selected from the group consisting of testosterone and methyltestosterone.

18. A method of producing a pressure-sensitive adhesive transdermal drug delivery system suitable for delivering a therapeutically effective amount of the one or more active agents at a substantially zero-order kinetic rate of delivery to the skin or mucosa of a patient in need thereof over a period in excess of 24 hours, comprising the steps of:
producing a blend of:
a therapeutically effective amount of one or more active agents;
one or more pharmaceutically acceptable adhesives; and
a rosin ester in an amount up to 25% by weight of the total composition, said amount being sufficient to suppress crystal formation of the one or more active agents during storage of the system,
forming the blend into a pressure-sensitive adhesive carrier composition wherein said composition excludes 1-menthol, and
drying the pressure-sensitive adhesive carrier to remove solvents to form the transdermal drug delivery system.
